(19) United States
(12) Patent Application Publication
(10) Pub. No.: US 2018/0042484 A1
(43) Pub. Date: Feb. 15, 2018

(54) PORTABLE, DURABLE, RUGGED, FUNCTIONAL NEAR-INFRARED SPECTROSCOPY (fNIRS) SENSOR

(71) Applicant: Charles River Analytics, Inc., Cambridge, MA (US)

(72) Inventors: Bethany K. Bracken, Cambridge, MA (US); Noa Palmon, Cambridge, MA (US); Seth Elkin-Frankston, Cambridge, MA (US); Filipe Silva, Lisbon (PT)

(21) Appl. No.: 15/673,934

(22) Filed: Aug. 10, 2017

Related U.S. Application Data
(60) Provisional application No. 62/373,717, filed on Aug. 11, 2016.

(51) Int. Cl. A61B 5/00; G01J 3/10; G01J 3/02

(52) U.S. Cl. CPC A61B 5/0075; G01J 3/0272; G01J 3/108; A61B 5/6814; A61B 5/0015

(57) ABSTRACT

A functional near-infrared spectroscopy sensor may include: a pliable substrate; a near infrared LED embedded in the pliable substrate; a red LED embedded in the pliable substrate; an optical detector embedded in the pliable substrate; a data storage device that receives and stores information derived from the optical detector and that is configured to attach to a head of a mammal or to an object that attaches to the head of mammal; and a source of electrical energy that powers the LEDs and the data storage device and that is configured to attach to a head of a mammal or to an object that attaches to the head of mammal.

### CROSS-REFERENCE TO RELATED APPLICATION

This application is based upon and claims priority to U.S. Provisional Patent Application No. 62/373,717, entitled “Portable, Durable, Rugged, Functional Near-Infrared Spectroscopy (fNIRS) Device,” filed Aug. 11, 2016, attorney docket number 75426-50. The entire content of this application is incorporated herein by reference.

### STATEMENT REGARDING FEDERALLY SPONSORED RESEARCH

This invention was made with government support under government contract numbers C13186, C14046, C13296, P16202, and C17057 with the United States Air Force and the United States Army (the Congressionally Directed Medical Research Programs; CDMRP). The government has certain rights in the invention.

### BACKGROUND

### Technical Field

This disclosure relates to functional, near-infrared spectroscopy sensors (fNIRS) and to their attachment to a user and use.

### Description of Related Art

fNIRS sensors are a non-intrusive, ambulatory method for measuring blood flow and blood oxygenation in cortical brain regions. NIRS uses light emitted from a light-emitting diode (LED) within the red (650-700 nm) and near infrared (700-1000 nm) range of the electromagnetic spectrum and photodetectors (optical diodes; ODs) to measure reflected light. Light in the red and near IR ranges passes through skin and bone and is reflected out of the body at different degrees of attenuation (as measured by the photodetectors) based on time-varying properties of the tissue it encounters.

Because oxygenated and deoxygenated blood have distinct absorption coefficients in the red and the near IR range, changes in blood oxygenation and concentration can be detected similarly to functional magnetic resonance imaging (fMRI) techniques. Although the spatial coverage and resolution are not as high as fMRI (NIR light may only penetrate about a centimeter past the skull), fNIRS sensors have excellent temporal resolution and can measure the large amount of cerebral processing that occurs on the surface of the brain. See Tichauer, K. M., Hadway, J. A., Lee, T. Y., & Lawrence, K. S. (2006), Measurement of cerebral oxidative metabolism with near-infrared spectroscopy: a validation study; Journal of Cerebral Blood Flow & Metabolism, 26(5), 722-730.; Keller, E., Nadler, A., Alkadhi, H., Kollias, S. S., Yonekawa, Y., & Niederer, P. (2003), Noninvasive measurement of regional cerebral blood flow and regional cerebral blood volume by near-infrared spectroscopy and indocyanine green dye dilution; Neuroimage, 20(2), 828-839.)

One example of a cognitive state measurable by fNIRS sensors is cognitive workload. Task performance is best at an optimum level of load. If a task is too easy or too difficult, performance decreases due to boredom or cognitive overload. Similarly, prefrontal cortical activation follows this same inverted U-shaped curve. During realistic tasks, such as the Warship Commander Task (Bunce, S., Izzetoglu, K., Ayaz, H., Shewokis, P., Izzetoglu, M., Pourrezaei, K., & Onaral, B. (2011), Implementation of fNIRS for monitoring levels of expertise and mental workload. Foundations of augmented cognition. Directing the future of adaptive systems, 13-22.), unmanned aerial vehicle piloting tasks (Ayaz, H., cakir, M. P., Izzetoglu, K., Curtin, A., Shewokis, P. A., Bunce, S. C., & Onaral, B. (2012, March). Monitoring expertise development during simulated UAV piloting tasks using optical brain imaging. In Aerospace Conference, 2012 IEEE (pp. 1-11). IEEE.), and air traffic control tasks (Ayaz, H., Shewokis, P. A., Bunce, S., Izzetoglu, K., Willems, B., & Onaral, B. (2012). Optical brain monitoring for operator training and mental workload assessment, Neuroimage, 59(1), 36-47.), fNIRS sensors have been used to measure the initial increase in the flow of oxygenated blood to the dorso-lateral prefrontal cortex that occurs as workload increases, as well as the decrease in blood flow to this region, as individuals disengage once task difficulty increases beyond their capacity to perform.

However, current research-grade fNIRS sensors can be obtrusive (requiring, at minimum, multiple light sources and detectors positioned across the forehead) and need to be wired to a large power source that must be ported along with the individual.

### SUMMARY

A functional near-infrared spectroscopy sensor may include: a pliable substrate; a near infrared LED embedded in the pliable substrate; a red LED embedded in the pliable substrate; an optical detector embedded in the pliable substrate; a data storage device that receives and stores information derived from the optical detector and that is configured to attach to a head of a mammal or to an object that attaches to the head of mammal; and a source of electrical energy that powers the LEDs and the data storage device and that is configured to attach to a head of a mammal or to an object that attaches to the head of mammal.

The LEDs, optical detector, data storage device, and source of electrical energy may be in a common housing that has a configuration that attaches to the head of the mammal or to the object that attaches to the head of mammal.

The optical detector may be shielded from light from the surrounding environment and from direct light from the LEDs.

The pliable substrate may be made of silicon.

The pliable substrate may have a surface and the light-emitting axis of the LEDs may be at an angle of about 45 degrees with respect to this surface.

The LEDs may be spaced apart by between 74-80 mm.

The current through the LEDs may be regulated by an API.

The intensity of the LEDs may be normalized to their peak intensity.

The optical detector may have a maximum wavelength sensitivity of 850 nm.

The optical detector may have a range sensitivity of 430 nm-1100 nm.

The pliable substrate may have a configuration that attaches to a head of a mammal or to an object that attaches to the head of mammal.

The sensor may have a configuration that transmits data in real time to a secondary device or database.

The sensor may record data at a preset time and for a preset duration.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps.

FIG. 1 illustrates an example of a suitable arrangement of fNIRS sensors.

FIG. 2 illustrates an example of the LEDs 101, the optical detector 103, and the 3D accelerometer 107 embedded within a pliable substrate 201, as well as a magnet 203.

FIG. 3 illustrates an example of the components illustrated in FIG. 2 connected to a hub 301.

FIGS. 4A-4C illustrate an example of a process of a user 401 attaching the components of the system illustrated in FIG. 4 to the user.

FIGS. 5A, 5B, 5C, and 5D, illustrate an isometric, side, top, and front view, respectively, of another example of the pliable substrate 201, having the LEDs 101, the optical detector 103, the 3D accelerometer 107, and the ferrous material 205.

FIG. 6 illustrates an example of an infrared spectrum that the infrared LED 101 may emit.

FIG. 7 illustrates an example of a red spectrum that the red LED 101 may emit.

FIG. 8 illustrates an example of a spectral intensity that the optical detector 103 may have.

FIG. 9 illustrates an example of a raw data that may be generated by the reflections from the red LED 101.

FIG. 10 illustrates an example of a raw data that may be generated by the reflections from the infrared LED 101.

FIG. 11 illustrates a schematic of an example of some of the circuitry that may be used for the sensor illustrated in FIG. 1.

### DETAILED DESCRIPTION OF ILLUSTRATIVE EMBODIMENTS

Illustrative embodiments are now described. Other embodiments may be used in addition or instead. Details that may be apparent or unnecessary may be omitted to save space or for a more effective presentation. Some embodiments may be practiced with additional components or steps and/or without all of the components or steps that are described.

fNIRS sensors are now described that can provide consistent readings, without intruding on the activity of the wearer. They may have a form factor (e.g., for a hat or military-style standard-issue helmet) that is mobile and comfortable to wear. The fNIRS sensors can be portable, durable, and rugged.

The fNIRS sensors may be portable, durable, and rugged enough to use in real-world environments, while individuals are participating in typical daily activities. Part of the sensor that contacts a person's forehead may be made of comfortable flexible material, such as silicon, with a magnetic backing or insert, so that it can be magnetically secured to a headband or hat.

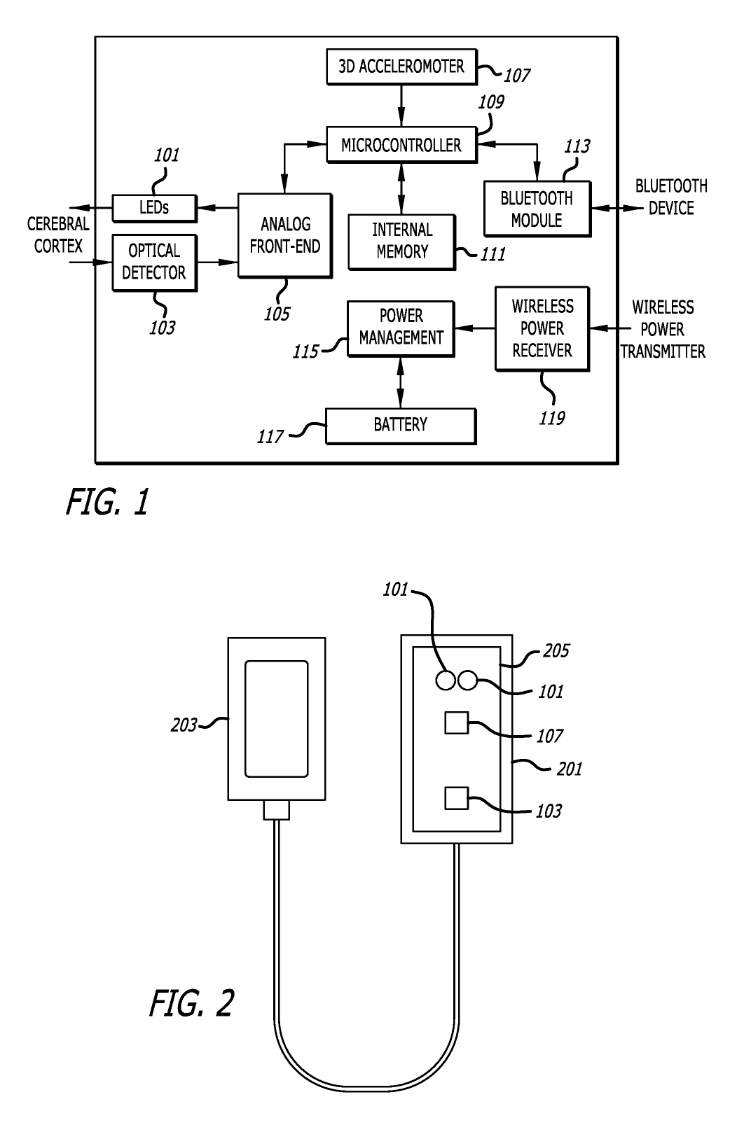

FIG. 1 illustrates an example of a suitable arrangement of fNIRS sensors. As illustrated in FIG. 1, the fNIRS sensor may include multiple LEDs 101, an optical detector 103, an analog front-end 105, a 3D accelerometer 107, a microcontroller 109, an internal memory 111, a Bluetooth module 113, power management system 115, a battery 117, and a wireless power receiver 119. Other components may also be included, and one or more of these listed components may be omitted.

The LEDs 101 may include one LED in the red (650-700 nm) region and another in the infrared (700-1000 nm) region of the spectrum. The red LED may have a peak emission of 660 nm, a half intensity beam angle of ±18 deg, spectral bandwidth of 25 nm, and a power output of up to 7 mW. The infrared LED may have a peak emission of 860 nm, a half intensity beam angle±13 deg, a spectral bandwidth of 30 nm, and a radiant intensity of 500 mW/sr. One or more of these parameters may instead have other values.

The LEDs 101 may be positioned so as to direct their light into the skull or other part of a mammal, such as a human. The pliable substrate 201 may have a substantially flat surface, and the light-emitting axis of the LEDs 101 may be at an angle of about 40-50 degrees with respect to this surface, such as at 45 degrees.

The LEDs may be spaced between 74-80 mm apart, such as 77 mm apart.

The optical detector 103 may be configured to detect light from the LEDs 101 that is reflected by tissue within the mammal, such as by tissue under a skull. A shield or other mechanism may be included to preclude light from other sources from reaching the optical detector 103, such as light from the environment. The optical detector may have a maximum wavelength sensitivity of 850 nm and a range sensitivity of 400 nm-1100 nm.

The analog front-end 105 may compute the difference on the optical detector 103 output between when the LEDs 101 are on and when the LEDs 101 are off, so that the ambient light component can be eliminated from the optical detector 103 output. The ambient light component can be eliminated by subtracting from the optical detector 103 output when the LEDs 101 are on, the value from this output when the LEDs 101 are off (ambient light component only).

The analog front end 105 may drive the LEDs 101 with the current intensity and the pulse timing and duration set by the microcontroller 109; and amplify, filter, and eliminate ambient light component of the optical detector output, convert it to a digital signal, and send it to the microcontroller.

The analog front end 105 may generate a number of bits for each LED channel that depends on the resolution of an internal analog-to-digital converter (ADC), such as 8 or 16-bits.

The LEDs 101 may be centered about the optical detector 103 and positioned at a distance from the optical detector 103, such as 23 mm away.

The 3D accelerometer 107 may detect acceleration of the user. It may be attached to any part of the user, and can be used as a separate data source or in combination from the data obtained from the optical detector 103, such as for motion-targeting de-noising purposes.

The microcontroller 109 may control the analog front-end 105 and receive data from it; control the 3D accelerometer 109 and receive data from it; store and retrieve data from the internal memory 111; send and receive data from the Bluetooth module 103; run an internal real-time clock to implement scheduling functionality to allow the device to turn on and record data at a pre-specified time(s) and a pre-specified duration(s); and control power management. The microcontroller may be configured to cause data to be recorded in the internal memory 111 and/or to be broadcast by the Bluetooth module 113 in real time and/or to initiate this recordation and/or broadcasting at a preset time for a preset duration. Other types of circuitry may be used instead to accomplish the same results.

The internal memory 111 may be used to store the data acquired from the optical detector 103 and the 3D accelerometer 107 on the device itself.

The Bluetooth module 113 may be used to communicate information between the microcontroller and an external device to store and/or handle data, such as a computer or mobile device.

The battery 117 may be used to power all of the other components and may be rechargeable. In lieu of or in addition to the battery 117, a photocell or other power source may be used.

The power management system 115 may be used to generate the power that is needed to operate the other components; supply power to the system from the battery or from the wireless power receiver; charge the battery when wireless power is being received; and regulate system power supply voltage. The power management system 115 may receive power from the battery 117 and/or the wireless power receiver 119.

The wireless power receiver 119 may wirelessly receive power from a remote source. The wireless power receiver 119 may include one or more coils that may receive this power through electromagnetic radiation from the remote source that can wirelessly charge the device.

FIG. 2 illustrates an example of the LEDs 101, the optical detector 103, and the 3D accelerometer 107 embedded within a pliable substrate 201, as well as a magnet 203. The positions of these various components may be different than is illustrated. The magnet 203 may be used to attach the pliable substrate 201 to an article that is worn by a user, such as to a hat or helmet, by placing the article between the substrate 201 and the magnet 203. Ferrous material 205 may be embedded within or attached to the substrate 201 so as to cause the pliable substrate to be attracted to the magnet 203.

The pliable substrate 201 may have any dimensions, such as 20 mm×40 mm.

The LEDs 101 in FIG. 2 and the corresponding optical detector 103 may be embedded within the pliable substrate 201 such that they all face the surface against which the pliable substrate 201 is placed and have a clear optical pathway to that surface, without protruding from the surface of the pliable substrate 201.

The pliable substrate 201 may be made of any pliable material, including rubbers, such as silicone.

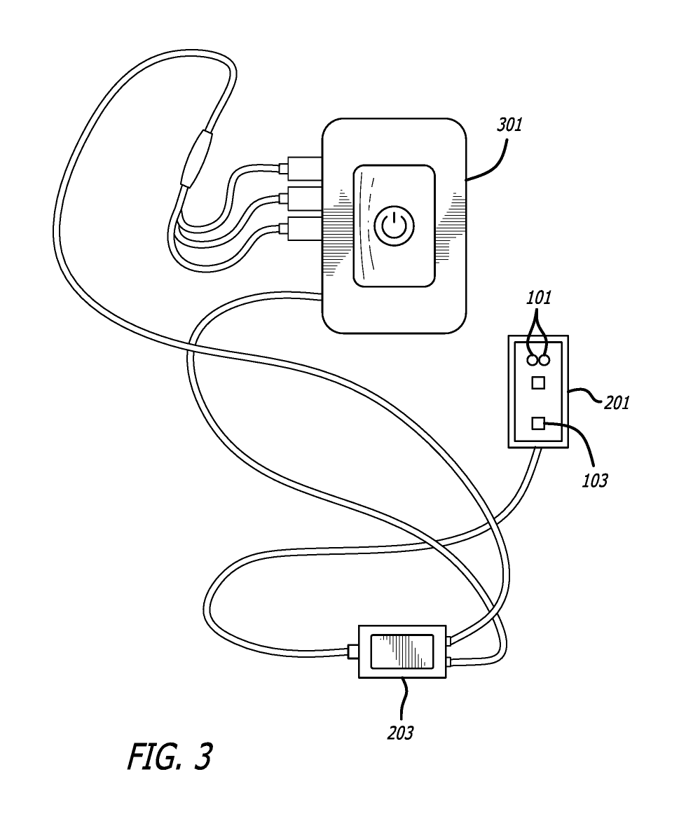

FIG. 3 illustrates an example of the components illustrated in FIG. 2 connected to a hub 301. As illustrated in FIG. 3, the LEDs 101 and optical detector 103 that are embedded in the substrate 201 may include wiring that connects to the hub 301. The magnet 203 may also be connected to both the substrate 201 and the hub 301 by cabling that ensures that the magnet 203 is not lost when detached. The hub 301 may contain the analog front end 105, the 3D accelerometer 107, the microcontroller 109, the internal memory 111, the Bluetooth module 113, the power management system 115, the battery 117, the wireless power receiver 119, other components, or any combination of these. The hub 301 may be configured to readily attach to a mammal or to an object (e.g., hat or helmet) that is attached to the mammal.

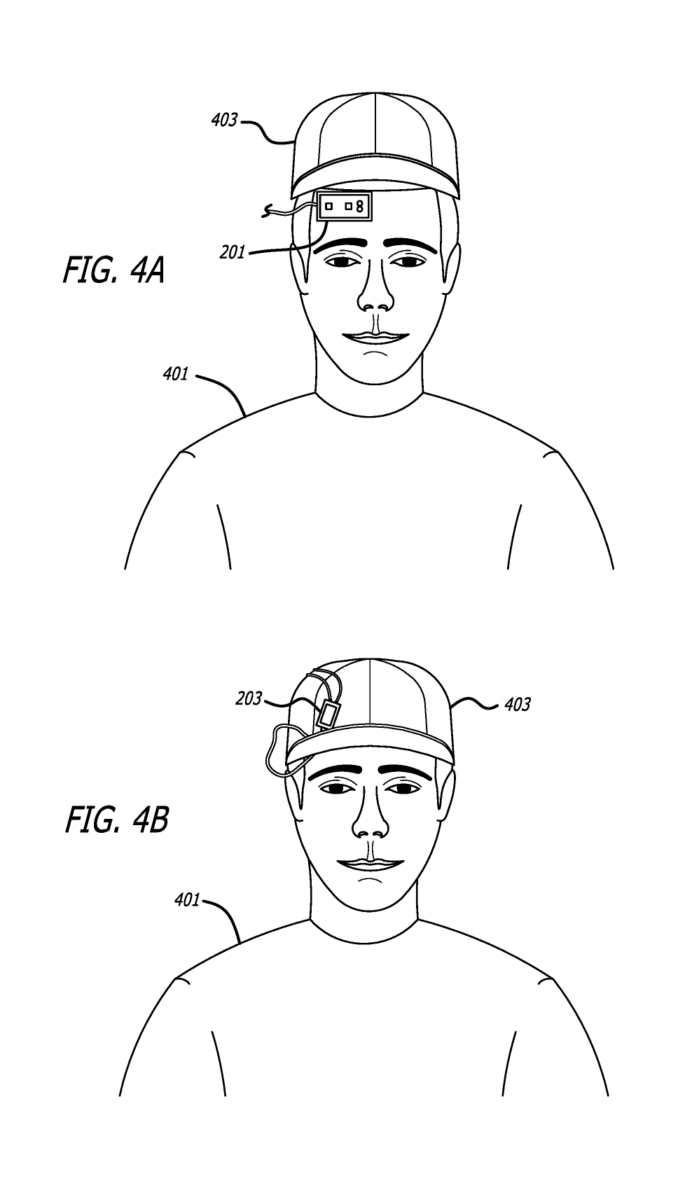

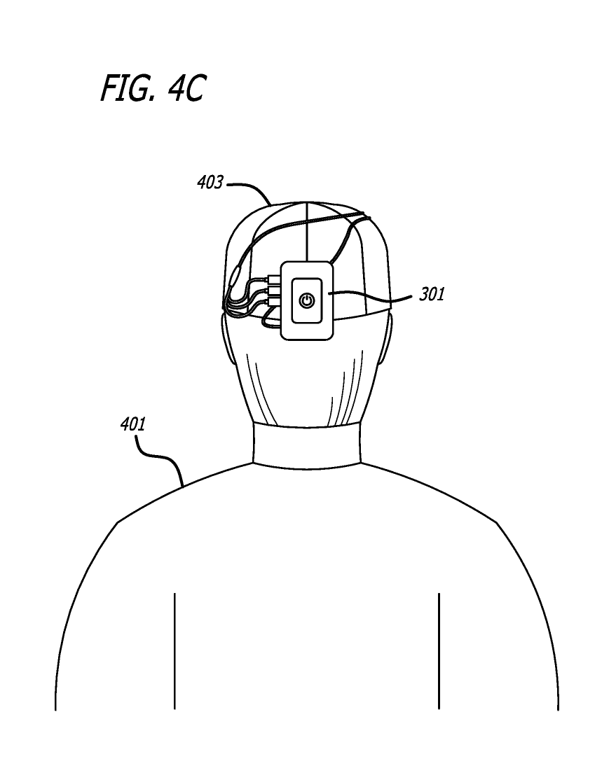

FIGS. 4A-4C illustrate an example of a process of a user 401 attaching the components of the system illustrated in FIG. 4 to the user. As illustrated in FIG. 4A, the user 401 may place the pliable surface 201 against a surface overlaying a portion of user tissue that is to be tested, such as against the forehead of the user 401. The user 401 may then slide a hat 403, helmet (not shown), strap (not shown), or other securing device (not shown) over the pliable substrate 201, helping to lock it in place, as illustrated in FIG. 4B. Adhesive tape or other means may in addition or instead be used. The user may place the magnet 203 on the opposite side of the hat 403 (or corresponding device) over the area of the flexible substrate 201 that contains ferrous material 205, thereby further securing the flexible substrate 201 in place, as also illustrated in FIG. 4B. The user 401 may then attach the hub 301 to the rear of the hat 403 or to another portion of the user 401 or to an article attached to the user 401, as illustrated in FIG. 4C.

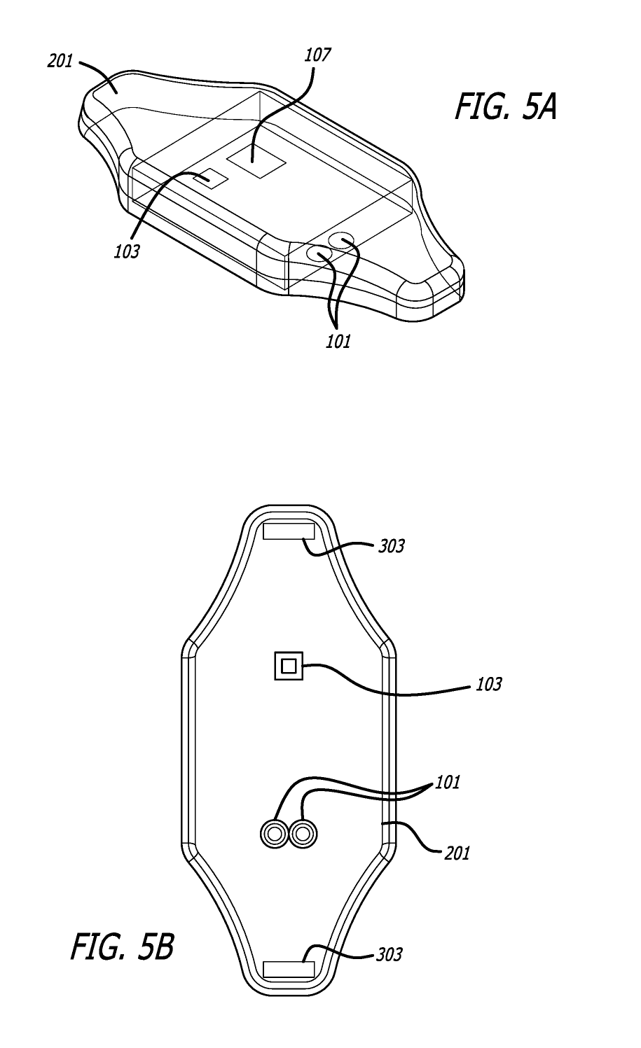

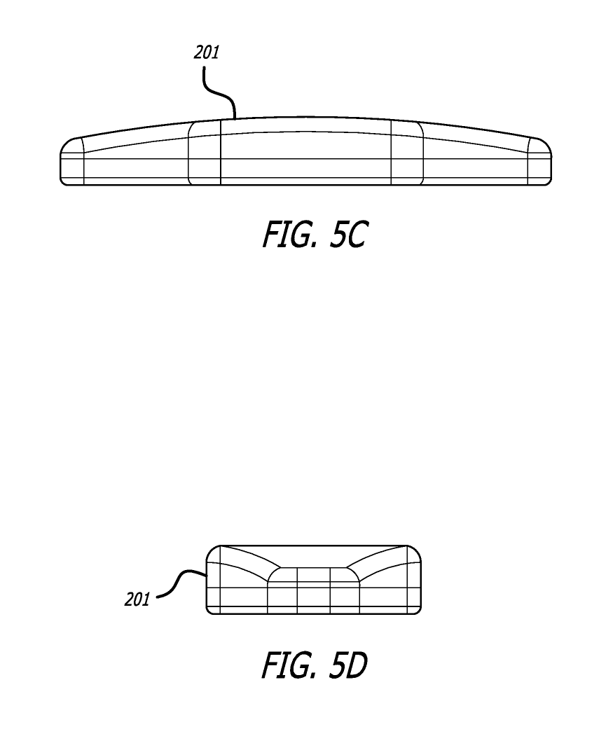

FIGS. 5A, 5B, 5C, and 5D, illustrate an isometric, side, top, and front view, respectively, of another example of the pliable substrate 201, having the LEDs 101, the optical detector 103, the 3D accelerometer 107, and the ferrous material 205. The shape and measurements that are shown are an example, and can be different.

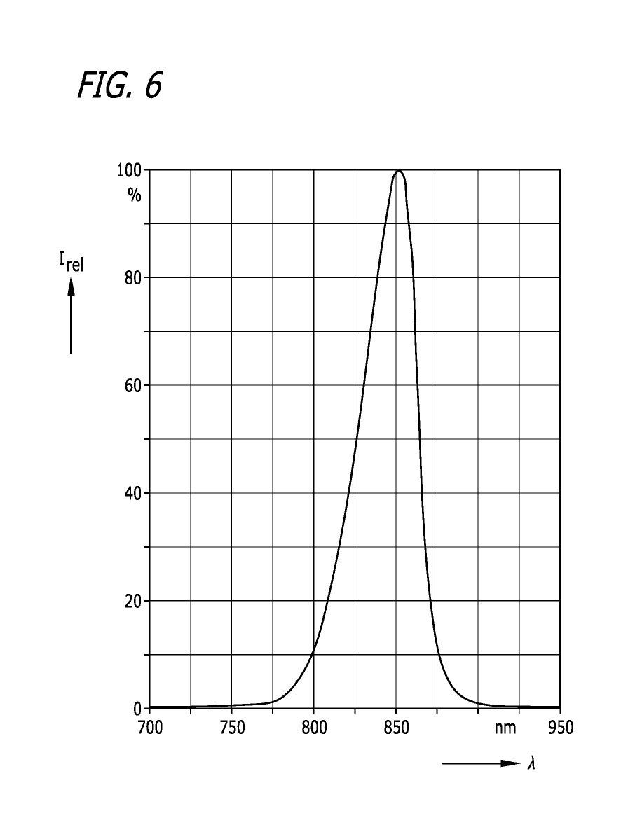

FIG. 6 illustrates an example of an infrared spectrum that the infrared LED 101 may emit. This may be when Irel=f(λ), TA=25° C., and IF=20 mA. The infrared LED may emit a different spectrum instead or have different operating conditions.

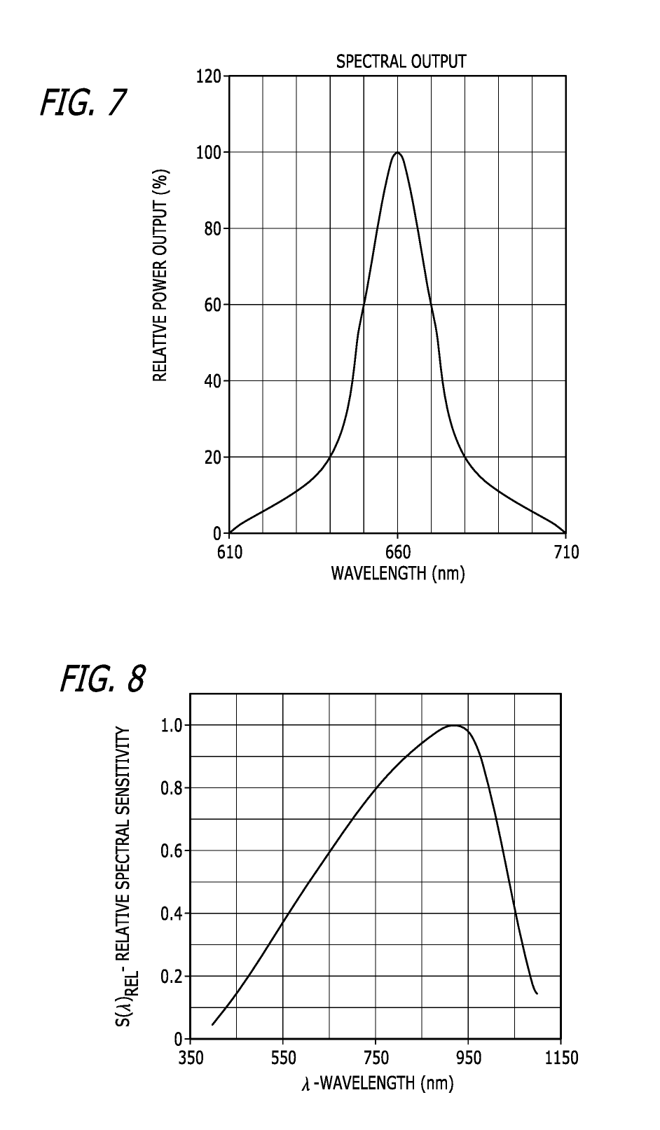

FIG. 7 illustrates an example of a red spectrum that the red LED 101 may emit. This may be when Irel=f(λ), TA=25° C., and IF=20 mA. The infrared LED may emit a different spectrum instead or have different operating conditions.

Sampling of the reflections from the LEDs may be, for example, at a frequency of 500 Hz, with a 25% duty cycle, and a resolution of 16 bits. The transfer function for both LEDs 101 may be:

[0 μA, 0.15 μA],

with

Current (μA)=(0.15*ADC)/2ⁿ,

where Current (μA) is the photodiode current in the optical detector 103 in microamperes (μA), ADC is the value sampled from the channel, and n is the number of bits of the channel.

FIG. 8 illustrates an example of a spectral intensity that the optical detector 103 may have. The optical detector 103 may have a different spectral intensity instead.

The optical detector 103 may have a wavelength of max sensitivity of 900 nm, a range of sensitivity of 430 nm-1100 nm, and a radiant sensitive area of 7.5 (mm2). The detector relative spectral sensitivity as shown in FIG. 4 is S rel=f(λ), TA=25° C.

FIG. 9 illustrates an example of a raw data that may be generated by the reflections from the red LED 101. Other raw data may be generated instead. The red LED current may be adjustable using an API to optimize performance.

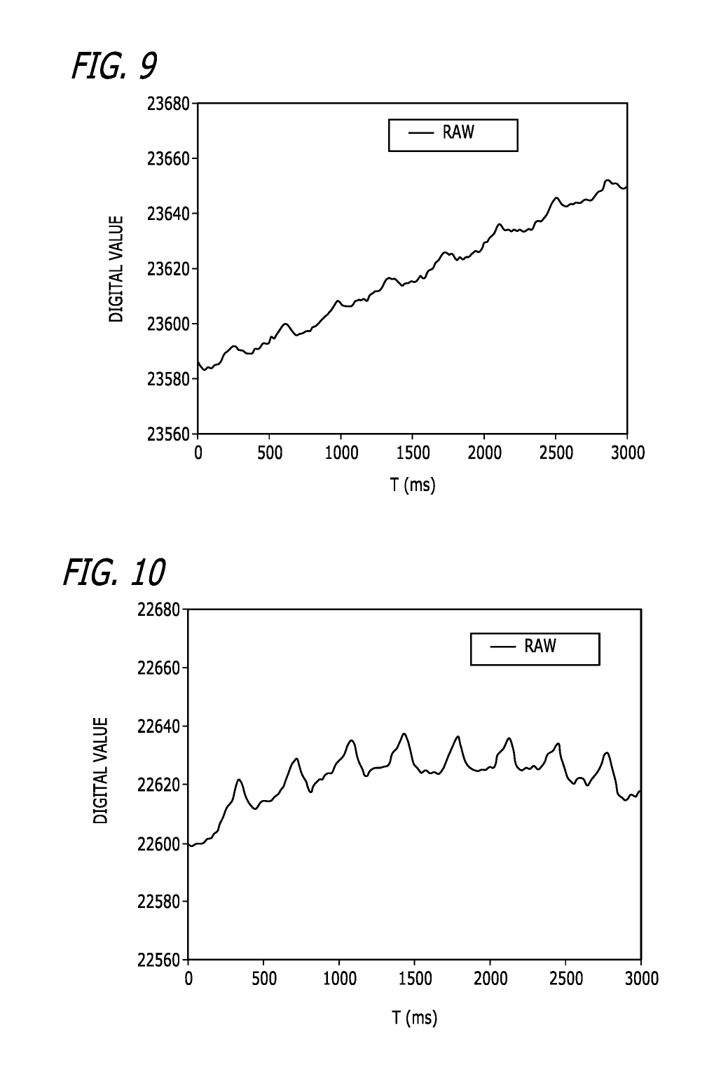

FIG. 10 illustrates an example of a raw data that may be generated by the reflections from the infrared LED 101. Other raw data may be generated instead. The infrared LED current may be adjustable using an API to optimize performance.

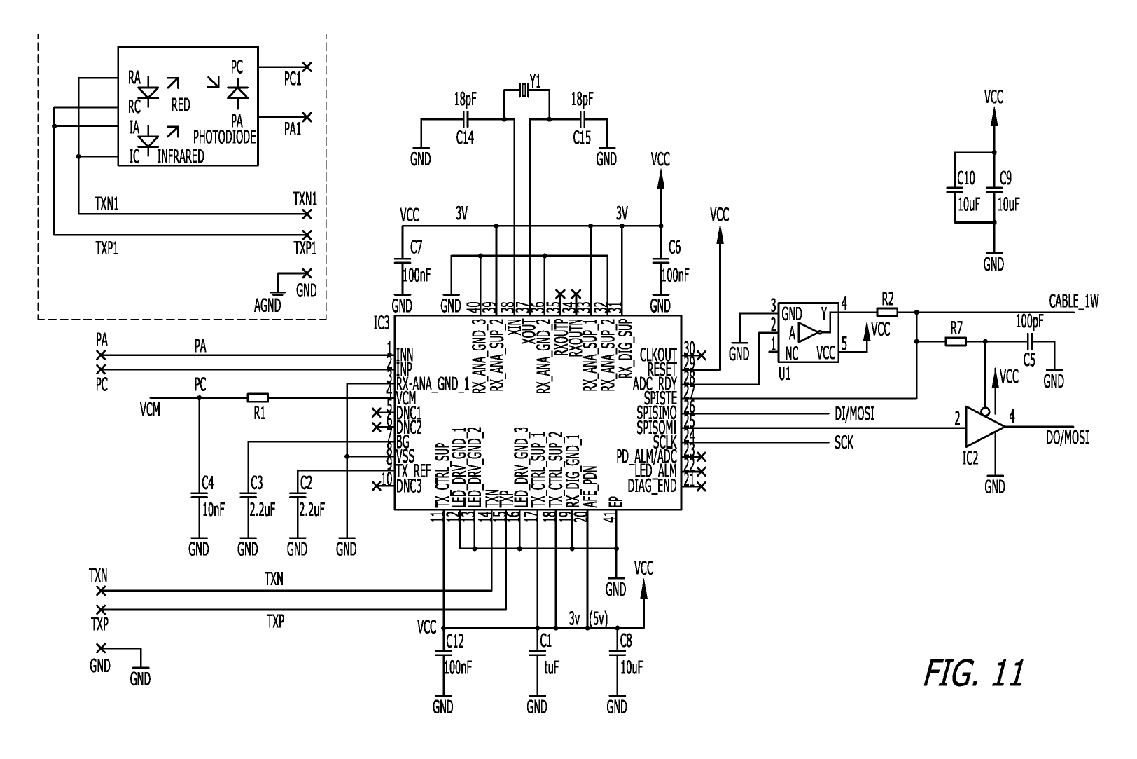

FIG. 11 illustrates a schematic of an example of some of the circuitry that may be used for the sensor illustrated in FIG. 1.

Applications for the fNIRS sensors that have been described may include oximetry, quantification of cardiac variables (e.g., heart rate, heart rate variability), life sciences studies, and biomedical research.

For example, the optical detector 103 (which may be a photodiode) may detect the reflected light from each of the LEDs 101. The produced current may be converted into a digital value that is sent via a serial peripheral interface (SPI). This information may be used to estimate the oxygen saturation level of blood and extract heart rate by measuring the wavelength of light that is reflected back out of the body, since oxygenated blood reflects light at a different wavelength than deoxygenated blood.

The components, steps, features, objects, benefits, and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits, and/or advantages. These also include embodiments in which the components and/or steps are arranged and/or ordered differently.

For example, the sensor could include additional LEDs and optical detectors to cover additional area across the forehead of the user; LEDs and optical detectors at multiple spatial distances to acquire signals from different depths into the cortical tissue; LEDs of different light frequency or intensity to capture alternate properties of the tissue (e.g., capturing additional cardiac information and less blood oxygenation information); LEDs at different angles to affect the reflection off of cortical tissue; or the addition of other sensors such as electrooculography (EEG) or electroencephalography (EEG).

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

All articles, patents, patent applications, and other publications that have been cited in this disclosure are incorporated herein by reference.

The phrase “means for” when used in a claim is intended to and should be interpreted to embrace the corresponding structures and materials that have been described and their equivalents. Similarly, the phrase “step for” when used in a claim is intended to and should be interpreted to embrace the corresponding acts that have been described and their equivalents. The absence of these phrases from a claim means that the claim is not intended to and should not be interpreted to be limited to these corresponding structures, materials, or acts, or to their equivalents.

The scope of protection is limited solely by the claims that now follow. That scope is intended and should be interpreted to be as broad as is consistent with the ordinary meaning of the language that is used in the claims when interpreted in light of this specification and the prosecution history that follows, except where specific meanings have been set forth, and to encompass all structural and functional equivalents.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element proceeded by an “a” or an “an” does not, without further constraints, preclude the existence of additional elements of the identical type.

None of the claims are intended to embrace subject matter that fails to satisfy the requirement of Sections 101, 102, or 103 of the Patent Act, nor should they be interpreted in such a way. Any unintended coverage of such subject matter is hereby disclaimed. Except as just stated in this paragraph, nothing that has been stated or illustrated is intended or should be interpreted to cause a dedication of any component, step, feature, object, benefit, advantage, or equivalent to the public, regardless of whether it is or is not recited in the claims.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various embodiments to streamline the disclosure. This method of disclosure should not be interpreted as requiring claimed embodiments to require more features than are expressly recited in each claim. Rather, as the following claims reflect, inventive subject matter lies in less than all features of a single disclosed embodiment. Thus, the following claims are hereby incorporated into the detailed description, with each claim standing on its own as separately claimed subject matter.

The invention claimed is:

1. A functional near-infrared spectroscopy sensor comprising:
a pliable substrate;
a near infrared LED embedded in the pliable substrate;
a red LED embedded in the pliable substrate;
an optical detector embedded in the pliable substrate;
a data storage device that receives and stores information derived from the optical detector and that is configured to attach to a head of a mammal or to an object that attaches to the head of mammal; and
a source of electrical energy that powers the LEDs and the data storage device and that is configured to attach to a head of a mammal or to an object that attaches to the head of mammal.

2. The sensor of claim 1 wherein the LEDs, optical detector, data storage device, and source of electrical energy are in a common housing that has a configuration that attaches to the head of the mammal or to the object that attaches to the head of mammal.

3. The sensor of claim 1 wherein the optical detector is shielded from light from the surrounding environment and from direct light from the LEDs.

4. The sensor of claim 1 wherein the pliable substrate is made of silicon.

5. The sensor of claim 1 wherein the pliable substrate has a surface and the light-emitting axis of the LEDs are at an angle of about 45 degrees with respect to this surface.

6. The sensor of claim 1 wherein the LEDs spaced apart by between 74-80 mm.

7. The sensor of claim 1 wherein current through the LEDs is regulated by an API.

8. The sensor of claim 1 wherein the intensity of the LEDs is normalized to their peak intensity.

9. The sensor of claim 1 wherein the optical detector has a maximum wavelength sensitivity of 850 nm.

10. The sensor of claim 1 wherein the optical detector has a range sensitivity of 430 nm-1100 nm.

11. The sensor of claim 1 wherein the pliable substrate has a configuration that attaches to a head of a mammal or to an object that attaches to the head of mammal.

12. The sensor of claim 1 having a configuration that transmits data in real time to a secondary device or database.

13. The sensor of claim 1 that records or transmits data at a preset time for a preset duration.
